(19) United States
(12) Patent Application Publication
(10) Pub. No.: US 2014/0234831 A1
(43) Pub. Date: Aug. 21, 2014

(54) Packaging or Other Materials Comprising a Biosensor and Methods of Their Use

(71) Applicant: CIRLE, Inc., Miami, FL (US)

(72) Inventor: Richard Awdeh, Miami, FL (US)

(21) Appl. No.: 14/347,682

(22) PCT Filed: Sep. 28, 2012
(86) PCT No.: PCT/US2012/057851

§ 371 Date: Mar. 27, 2014

Related U.S. Application Data
(60) Provisional application No. 61/541,432, filed on Sep. 30, 2011.

(51) Int. Cl. C12Q 1/10; C12Q 1/04; C12Q 1/14

(52) U.S. Cl. CPC C12Q 1/10; C12Q 1/14; C12Q 1/04
USPC 435/5; 435/288.7; 435/287.9; 435/287.7; 435/34; 435/7.32; 435/38; 435/36; 435/7.31

(57) ABSTRACT

Provided herein is a composition for detection of a microbial product in a sample comprising a polydiacetylene (PDA) and a packaging polymer, wherein the sample contacts the PDA and a change of PDA color indicates detection. Also provided herein are methods of making a packaging material comprising a PDA and methods of using the composition for detection of a microbial product in a sample.

### CROSS-REFERENCE TO RELATED APPLICATIONS

The present application claims the priority benefit of U.S. Provisional Patent Application No. 61/541,432 filed Sep. 30, 2011.

### BACKGROUND

A biosensor is an analytical device that employs biological elements such as enzymes, antibodies, nucleic acids, and microorganisms for their specific biological interactions with target items. For detection, various methods such as colorimetric detection, fluorescent detection, and electrochemical detection have been used. Colorimetric detection is the easiest and the most convenient method because detection can be done using the naked eye. Biosensors offer advantages as alternatives to conventional analytical methods because of their inherent specificity, simplicity, and quick response.

Polydiacetylene (PDA) is widely known because of its unique optical properties. The PDA polymer is formed by the 1,4 addition of diacetylenic monomers, which is initiated by ultraviolet irradiation. The result is an intensely colored polymer, typically of a deep blue color. Among the first demonstrations of potential PDA biological applications was the colorimetric detection of the influenza virus, which relied on the reaction between the derivatized diacetylenic monomer and the cellular receptor of the virus [Charych et al., 1993. Science. 261(5121), 585-588].

### BRIEF DESCRIPTION OF THE DRAWINGS

FIG. 1 is a schematic representation of lamellar PDA domains associated with/within a sol-gel, packaging polymer, or sol-gel packaging polymer matrix. (A. matrix, B. PDA domains, C. PDA domains associated with matrix)

FIG. 2 shows microscopy images of lamellar PDA domains on a sol-gel matrix.

FIG. 3 contains pictures showing sol-gel/PDA patches and coated plastic tubings with color changes induced by bacteria.

FIG. 4 is a schematic of the creation of packaging polymer/PDA thin sensor films at the air/water interface.

FIG. 5 is a schematic of the morphology of the packaging polymer/PDA films created at the air/water interface. (A. PDA lamellar domains, B. Polymeric matrix, C. PDA lamellar domains and polymeric matrix at the air/water interface.)

FIG. 6 is a schematic of the process in which lipid/PDA vesicles are encapsulated within a porous transparent matrix and used for microbial detection.

### DETAILED DESCRIPTION

Provided herein is a composition for detection of a microbial product in a sample comprising a biosensor, i.e., polydiacetylene (PDA), and a packaging or other polymer, wherein the sample contacts the biosensor. In some embodiments, the biosensor is PDA and a change of PDA color indicates detection. PDA compositions are used because when the PDA monomers crosslink, they appear an intense blue color owing to their conjugated ene-yne framework [Reppy M A, and Pindzola B A. 2007. Chem. Commun., 4317-4338]. External structural perturbations, such as binding of amphiphilic and bacterial membrane associated hydrophobic molecules causes conformational transitions in the conjugated polymer backbone leading to intense blue-red color changes [Okada, S., R. Jelinek, and D. Charych. 1998. Angew. Chem. Int. Ed. Engl. 38:655-659; Kolusheva, S., L. Boyer, and R. Jelinek. 2000. Nat. Biotechnol. 18:225-227; Silbert L, Shlush I B, Israel E, Porgador A, Kolusheva S, Jelinek R. 2006. Rapid Chromatic Detection of Bacteria by Use of a New Biomimetic Polymer Sensor. Applied and Environmental Microbiology. 72: 7339-7344].

Accordingly, provided herein are packaging material compositions or other polymeric materials that comprise a PDA polymer. The packaging or other polymeric material can comprise the PDA via incorporation into the packaging material, via coating of the packaging material, or via attachment to the packaging material. In some embodiments, a packaging or other polymeric material is provided that has a PDA attached thereto in the form of a sticker, agarose gel, or other PDA coated or impregnated material. Accordingly, also provided herein are methods of making a packaging or other polymeric material comprising a PDA.

Definitions

As used in the specification and claims, the singular form “a”, “an” and “the” include plural references unless the context clearly dictates otherwise. For example, the term “a polymer” includes a plurality of polymers, including mixtures thereof.

“Aliphatic group” refers to a saturated or unsaturated, linear or branched hydrocarbon group and encompasses alkyl, alkenyl, and alkynyl groups, for example.

“Alkyl” refers to a monovalent group derived from a straight or branched chain saturated hydrocarbon by the removal of a single hydrogen atom. Exemplary alkyl groups include methyl, ethyl, n- and iso-propyl, cetyl, and the like.

“Alkylene” refers to a divalent group derived from a straight or branched chain saturated hydrocarbon by the removal of two hydrogen atoms. Exemplary alkylene groups include methylene, ethylene, propylene, and the like.

“Amido group” and “amide” refer to a group of formula —C(O)NY1Y2, where Y1 and Y2 are independently selected from H, alkyl, alkylene, aryl and arylalkyl.

“Amino group” and “amine” refer to a group of formula —NY3Y4, where Y3 and Y4 are independently selected from H, alkyl, alkylene, aryl, and arylalkyl.

“Amidoamine group” or “amidoamine” refer to compounds having an amine group and an amide group.

“Cycloalkyl” refers to a saturated alicyclic hydrocarbon such as cyclopropane, cyclobutane, cyclopentane, cyclohexane, and the like.

The terms “diacetylene” and “diacetylene monomer” refer to a chemical having the formula of CH₄H₂. The terms “polydiacetylene” and “PDA” refer to a composition containing two or more diacetylene monomers and having the chemical formula of I:

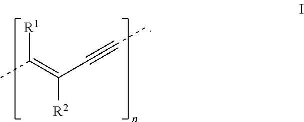

wherein R¹ and R2 are each independently selected from H, a C₁-C₁₂, or C₁-C₈, or C₁-C₆, or C₁-C₄ straight-chain or branched, or a C₃-C₁₂, or C₃-C₈, or C₃-C₆ cyclic, substituted or unsubstituted, alkyl radical, and wherein “n” is between 1 and 10,000. The polydiacetylenes provided herein include 10,12-tricosadiynoic acid, 5,7-pentacosadiynoic acid, 10,12-pentacosadiynoic acid, 10,12-pentacosadiynoate, and 5,7-docosadiynoic acid. A “polydiacetylene solution” or a “PDA solution” comprises a polydiacetylene as defined herein.

As used herein, the term “microbe” includes a bacterium, fungus, virus, protozoan, and yeast. Exemplary microbes include Serratia spp., Pseudomonas spp., Staphylococcus aureus, Staphylococcus pneumonia, and fusarium (fungi). A “microbial product” includes an enzyme or other composition secreted by a microbe.

The term “packaging material” is defined herein to include any material that can be used to package or contain liquids, animal products, and the like. In some embodiments, the “packaging material” comprises a plastic. “Other polymeric materials” include, but are not limited to, surgical gowns, surgical dressings, contact lenses, and medical devices.

As used herein, the term “packaging monomer” includes, but is not limited to, an ethylene, propylene, styrene, vinyl chloride, vinyl acetate, vinyl alcohol, vinylidene chloride, carbonate, amide, ethylene terephthalate, and ethylene-vinyl acetate. The term “packaging polymer” refers to a composition comprising two or more packaging monomers.

In some embodiments, a packaging or other polymeric material is provided that has a PDA incorporated therein. In these embodiments, a packaging or other monomer and a diacetylene monomer are mixed and polymerized prior to formation of the packaging or other material. “Formation” of a packaging or other material includes curing and molding the packaging material into a desired shape. A desired shape can be a bottle or other type of container. A desired shape can also be a medical device, contact lense, surgical gown or surgical dressing.

In preparing packaging or other polymeric materials having a PDA incorporated therein, diacetylene monomers and packaging or other monomers can be mixed in organic solvent/s, aqueous solutions, or mixtures. Parameters to be modulated are: solvent type, ratio between the monomers, and addition of additives required for plastic properties. Lipid molecules can also be added to stabilize the PDA. The diacetylene monomers and packaging or other monomers are then polymerized. Parameters to be modulated are separate polymerization of components/simultaneous polymerization; degree of polymerization; and polymerization before/after molding. The PDA and packaging or other polymers are then molded to the desired shape/structure and curing/annealing. Parameters to be modulated are: duration of curing; temperature; and post-curing polymerization steps.

In some embodiments, the packaging or other polymeric material comprises a packaging or other polymer, a PDA and a hydrolyzed silica. In these embodiments, a packaging or other monomer, a diacetylene monomer, and a silica precursor are mixed prior to formation of the packaging material. Silica precursors that can be used as described herein include, but are not limited to, tetraethyl orthosilicate (TEOS), tetramethyl orthosilicate (TMOS), methyltrimethoxysilane (MTMS), diethoxydimethylsilane (DEMS), and vinylotriethoxysilane (VTES).

In one example, diacetylene monomers are mixed with silica precursors. Parameters to be modulated are: ratios among components; type of silica precursors; and nature of solvents. Packaging monomers are then dissolved in appropriate solvents. Parameters to be modulated are polymer preparation protocols. The two monomer solutions are mixed. Parameters to be modulated are: timing of reagent addition and mixing; temperature; and ratios. The mixture is molded to desired shapes and structures, and cured and polymerized. Parameters to be modulated are: the order of the two processes; and duration. In some embodiments, a mixed assembly is created through thin film techniques (dip-coating, layer-by-layer, or spin coating).

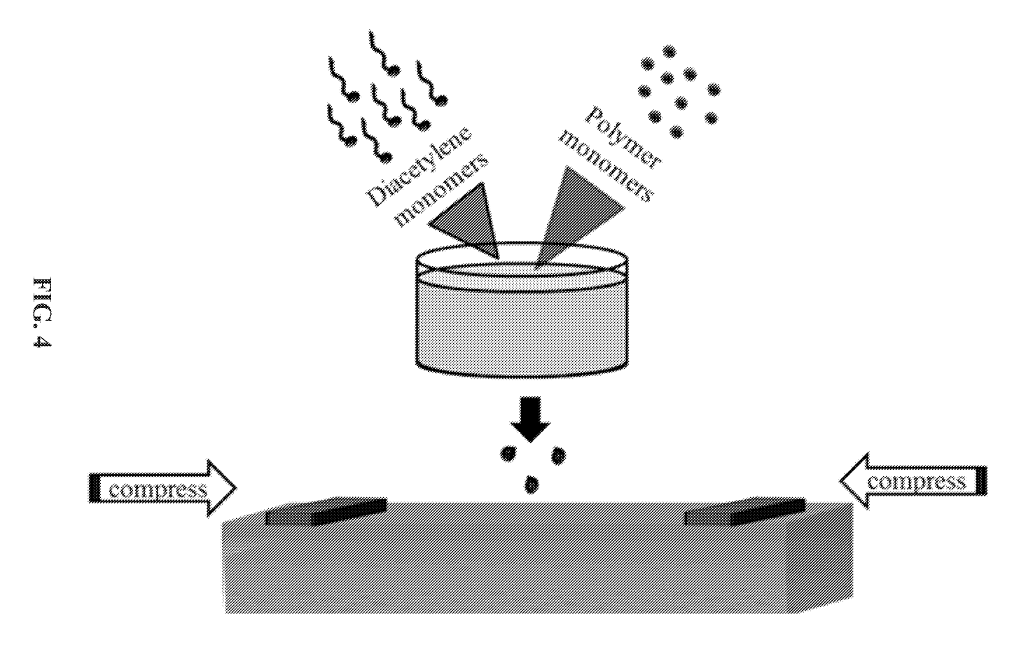

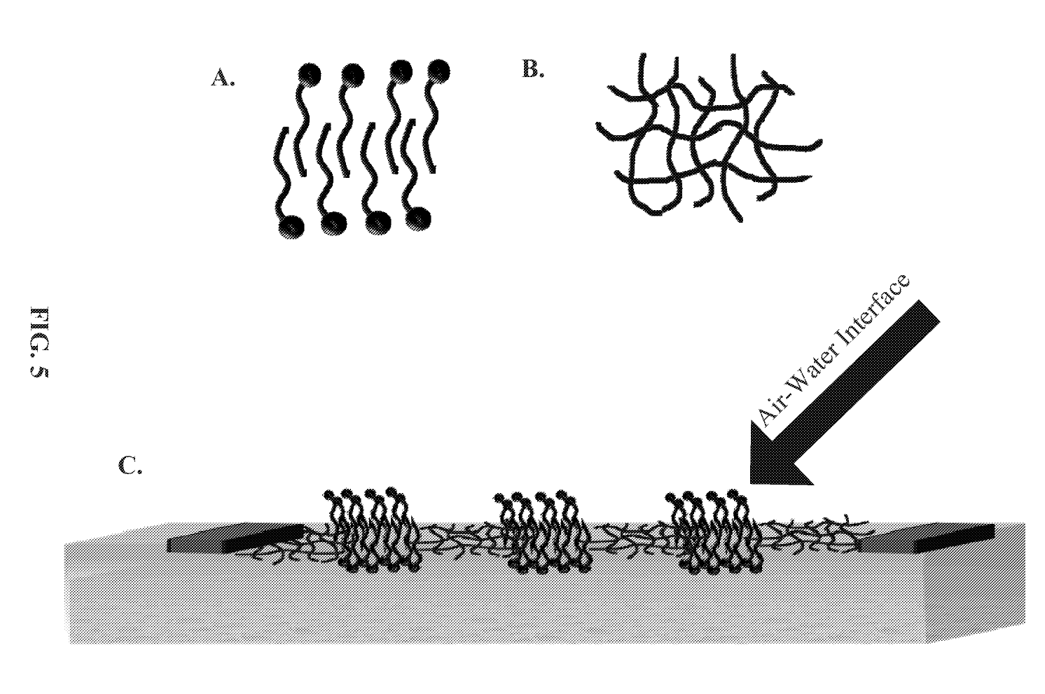

In one embodiment, a packaging or other polymeric material is made using a process comprising the steps of: 1) mixing a diacetylene monomer with a silica precursor (first solution), 2) mixing the first solution with a packaging or other monomer to form a second solution, and 3) polymerizing the second solution. In another embodiment, a packaging or other polymeric material is made using a process comprising the steps of: 1) mixing a diacetylene monomer, a silica precursor and a packaging or other monomer, and 2) polymerizing the mixture. Sol-gel/PDA films can also be prepared at the air/water interface, i.e. using the Langmuir method and/or a method generally shown in the schematics of FIGS. 4 and 5. These sol-gel/PDA films are then transferred onto the packaging or other substrate. Polymerization can be carried out prior to film transfer or after.

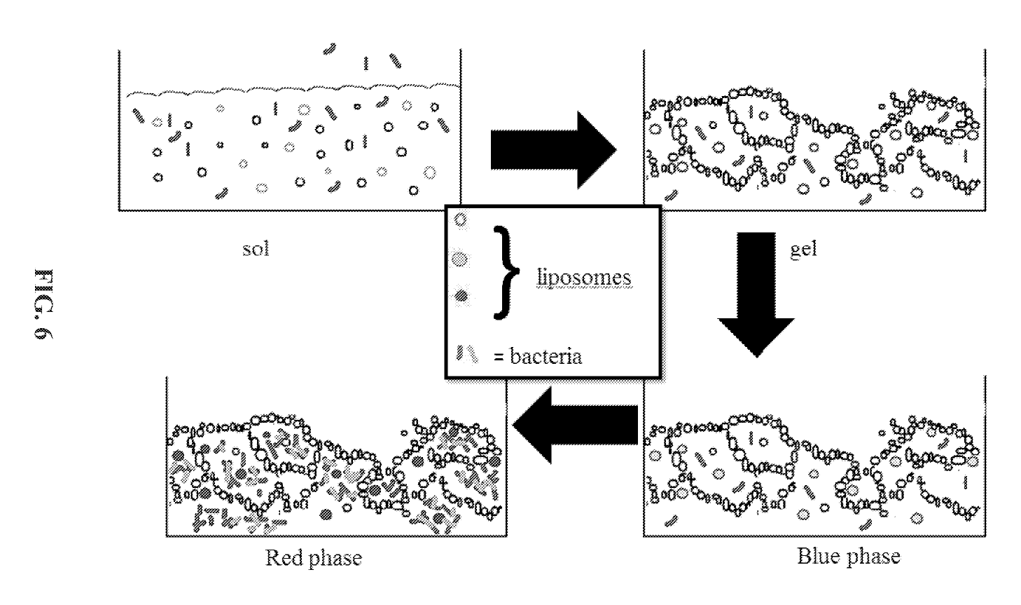

In some embodiments, the PDA polymer is contained within a vesicle and incorporated into a packaging or other polymeric material or coated upon a packaging or other polymeric material. Lipid/PDA vesicles that are embedded in a porous matrix and used for bacterial detection are described in U.S. Pat. No. 7,794,968 and U.S. Pat. No. 8,008,039. More particularly, in some embodiments, a packaging or other polymeric material is formed by a process comprising the steps of: 1) dissolution of a diacetylene monomer in an aqueous solution to result in formation of a PDA vesicle (first solution), 2) dissolution of a packaging or other monomer in a mild organic solvent (second solution), 3) mixing the first and second solutions to form a third solution, and 4) polymerizing the third solution. Examples 2 and 4 provided below also discuss such embodiments in further detail. In some other or further embodiments, the size of the pores in the porous matrix are regulated by forcing air into the mixture (or using some other chemical or physical means) prior to solidification. FIG. 6 provides a relevant schematic.

In some embodiments, a packaging or other polymeric material is provided that has a PDA solution coated thereupon. A PDA solution can be coated on the entire packaging material, or any portion of a container comprising the packaging material, or any portion of a medical device or other polymeric material, including, but not limited to, one side of a packaging material container, medical device, or other polymeric material, an interior portion of a packaging material container, medical device, or other polymeric material and a neck or lip portion of a packaging material container. In one embodiment, a PDA solution is coated onto the neck or lip portion of a liquid container such as a contact lens solution container. Also included herein is a packaging material having a PDA solution coated thereupon and further having an additional transparent protective coating.

The PDA solution can be coated via any means including, but not limited to, dip coating, aerosol coating, coating with monolayers prepared at the air/water interface, and PRINT (pattern replication in non-wetting templates) technology methods. [See M Ritenberg et al., ChemPlusChem, 2012, 77, in publication for dip coating methods.] Coating includes the application of a single layer of PDA solution, multiple layers of PDA solutions (identical or different PDA solutions), and multiple layers of PDA solutions and other solutions or materials. In some embodiments, a packaging material is coated with a PDA solution comprising 10,12-tricosadiynoic acid, tetraethyl orthosilicate, nitric acid, and water. The mole ratios of the 10,12-tricosadiynoic acid, tetraethyl orthosilicate, nitric acid, and water can be approximately 1:9:312:0.13:0.05, respectively.

In addition to a packaging or other polymeric material comprising a PDA, provided herein is a method for detecting one or more microbes in a sample by using the packaging material. More particularly, included herein is a method for detecting one or more microbes in a sample comprising, contacting the sample with a packaging or other polymeric material comprising a polydiacetylene (PDA), wherein a color change in the PDA indicates detection. In some embodiments, the sample is disposed within a container, the container comprises a wall, an interior, and a cap, and the interior container wall comprises the PDA. In other embodiments, the PDA is directly within the plastic material of a medical device, i.e., an intraocular lens (IOL) that is then implanted into the eye. The packaging or other polymeric material can comprise the PDA via incorporation into the packaging or other polymeric material, via coating of the packaging or other polymeric material, or via attachment to the packaging or other polymeric material. The packaging or other polymeric materials used in the methods described herein can be any of those described above or below.

It should be understood that the foregoing relates to preferred embodiments of the present disclosure and that numerous changes may be made therein without departing from the scope of the disclosure. The disclosure is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present disclosure and/or the scope of the appended claims. All patents, patent applications, and publications referenced herein are incorporated by reference in their entirety for all purposes.

### EXAMPLES

### Example 1

### Preparation of PDA/Packaging Polymer Materials by Mixing Diacetylene Monomers and Packaging Monomers

In one embodiment, the packaging monomer is prepared by coating a Silicon wafer or glass substrate with (tridecafluoro-1,1,2,2-tetrahydrooctyl)trichlorosilane by keeping the substrate and a drop of the reagent kept in a vial in a desiccator for 30 minutes. First, the base is mixed with the curing agent at a 10:1 ratio by weight. Air bubbles are then removed from the mixture by applying a vacuum and the mixture is poured on the substrate. The resultant silicon or glass monomer is then placed in an oven maintained at 700° C. for 2 hours to make it solidified.

In parallel, PDA is prepared by evaporating 140 ml of diacetylene monomer for at least 4 hours at 60 mbar conditions. 2 mL of DDW (doubly distilled water) is then added to the monomer solution. The mixture is sonicated using intervals for 4 minutes at 70° C. and then cooled to room temperature. The PDA mixture and the silicon or glass polymer are then mixed and cured. Polymerization of PDA is subsequently carried out through exposure of the material to ultraviolet light (254 nm) for several seconds, until it appears blue. In another embodiment, gel is substituted for the silicone or glass polymer.

### Example 2

### Preparation of PDA/Packaging Polymer Materials by Mixing Diacetylene Monomer Vesicles and Packaging Monomers

In some embodiments, diacetylene monomers are dissolved in aqueous solution and small particles/vesicles are constructed. Parameters to be modified are: concentration; pure diacetylene monomers or mixtures with lipids/surfactants/additives to enhance stability; and size of formed particles. Packaging monomers are dissolved in aqueous solution or mild organic solvents (mild—to prevent dissolution of diacetylene particles after mixing). The two solutions are mixed. Parameters to be modified are: ratios; duration before mixing; and degree of polymerization of individual solutions prior to mixing. The mixture is the polymerized. Parameters to be modified are: degree of polymerization; duration; and timing of polymerization (prior or after molding). Molding and curing to desired shapes is then performed.

### Example 3

### Preparation of PDA/Sol-Gel/Packaging Polymer Materials by Mixing Diacetylene Monomors, Silica Precursors and Packaging Monomers

In one embodiment, the packaging monomer is prepared by coating a Silicon wafer or glass substrate with (tridecafluoro-1,1,2,2-tetrahydrooctyl)trichlorosilane by keeping the substrate and a drop of the reagent kept in a vial in a desiccator for 30 minutes. First, the base is mixed with the curing agent at a 10:1 ratio by weight. Air bubbles are then removed from the mixture by applying a vacuum and the mixture is poured on the substrate. The resultant silicon or glass monomer is then placed in an oven maintained at 700° C. for 2 hours to make it solidified.

The sol-gel component is prepared by mixing tetramethoxysilane (TMOS), water and 0.62M HCL (4.41:2.16:0.06 v:v:v). The mixture is incubated for one hour with stirring at 4° C., diluted with water 1:1 (v:v) and then evaporated for approximately six minutes at 60 mbar. Then, after sonication in water, lipid/polydiacetylene (PDA) vesicles (PDA/DMPC 3:2, mole ratio) were prepared by dissolving the lipid components in chloroform/ethanol and drying together in vacuo. Vesicles were subsequently prepared in DDW by probe-sonication of the aqueous mixture at 70° C. for 3 min. The vesicle solution was then cooled at room temperature for an hour and kept at 4° C. overnight. 7 mM DMPC/PDA liposomes are diluted with Tris pH 7.5 1:1 (v:v). The solution of liposomes and the solution of silica gel are mixed 1:1 (v:v) and immediately placed in a 384-well ELISA plates (15 μl in each well). Gelation then occurs for 30 minutes at room temperature. After gelation, each well is filled with a Tris pH 7.5 solution for storing in a refrigerator. After a minimum of overnight in the refrigerator, the mixture is polymerized for 2 minutes before it is heated to room temperature (30 minutes).

The PDA/sol-gel mixture is then prepared as follows. 140 microliters of diacetylene/dimyristoylphosphatidylcholine (DMPC) total concentration 7 mM, mole ratio 3:2 (PDA:DMPC) is evaporated for at least 4 hours at 60 mbar conditions. 2 mL of DDW is then added to the diacetylene/DMCP solution and sonicated for 6 minutes (3 minutes with heat). After cooling to room temperature, the diacetylene/DMCP solution is mixed with the pre-solidified sol-gel component. The mixture is allowed to solidify and PDA is polymerized using ultraviolet irradiation at 254 nm. Packaging monomers are added to the mixture prior to PDA polymerization.

### Example 4

### Preparation of PDA/Silica Materials to be Coated onto Packaging Materials

Precursor solutions were synthesized from tetraethyl orthosilicate (TEOS), diacetylene (TRCDA, or 10,12-tricosadiynoic acid) and HNO₃ catalyst prepared in a tetrahydrofuran (THF)/water solvent at room temperature. The final reactant mole ratios were 1:9:312:0.13:0.05 (TRCDA:TEOS:THF:HNO₃:H₂O). After one day aging at ambient temperature, the silica/PDA sol solution was filtered through 0.45 μm nylon and kept at −200° C.

For deposition on a packaging material, the material to be coated was dipped in the silica/PDA sol and kept immersed for 1 minute. After this, the packaging material was pulled out at withdrawal speed of approximately 35 mm/s. Following air-drying, uniform thin films are ultraviolet-irradiated (254 nm) for 1 minute to produce the blue-phase PDA thin film material.

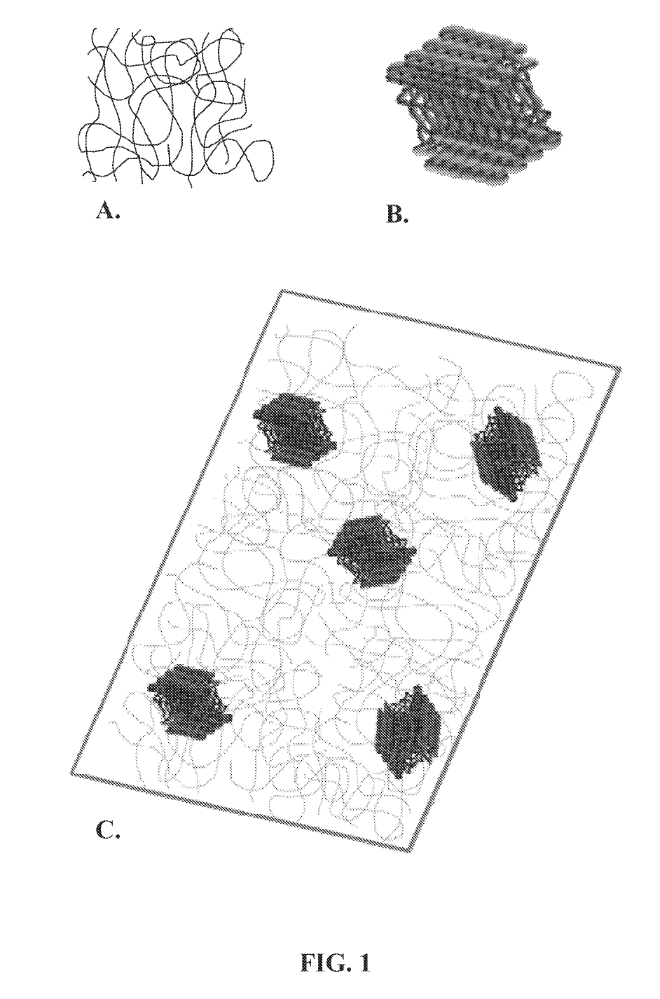

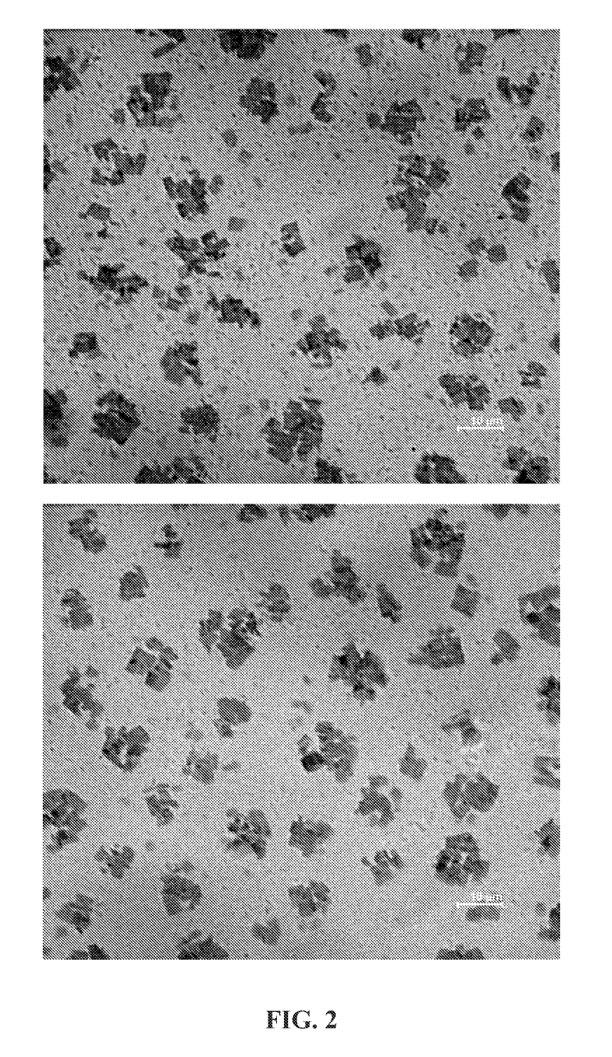

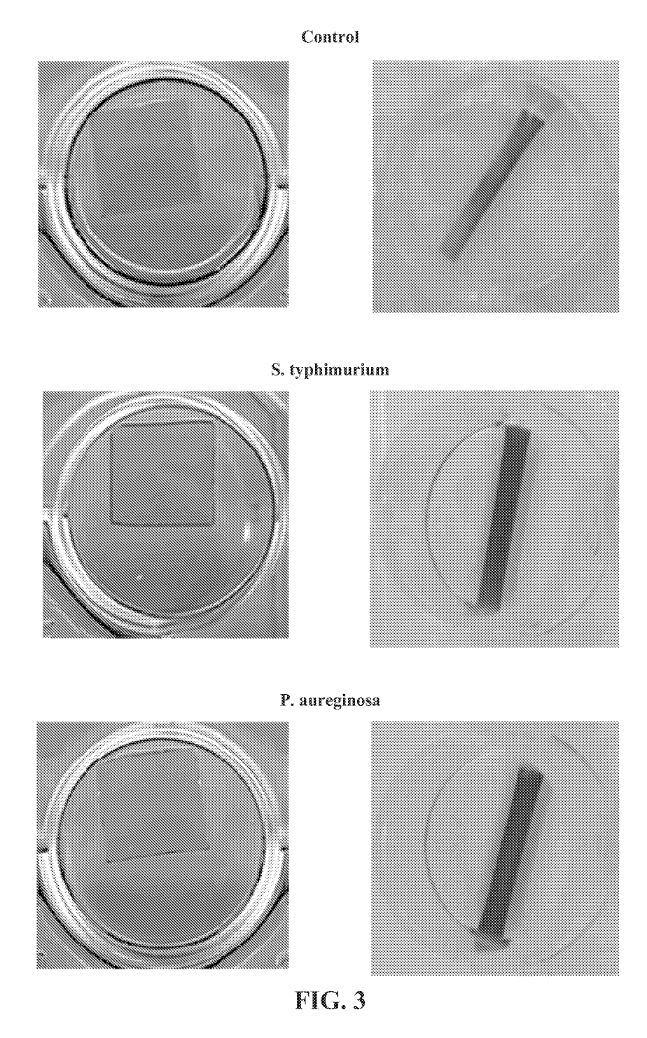

FIG. 1 shows a schematic of discrete diacetylene lamellar domains distributed across a sol-gel, packaging polymer, or sol-gel/packaging polymer surfaces. FIG. 2 shows microscopy images of discrete diacetylene lamellar domains distributed across a sol-gel surface. FIG. 3 shows the results of patches and tubing coated with the sol-gel/PDA solutions, which patches and tubing were subsequently contacted with either a control, S. typhimurium or P. aureginosa. This figure demonstrates that PDA solutions comprising silica can be coated onto packaging materials and used to detect microbes and/or microbial products.

### Example 5

### Preparation of PDA/Vesicle Materials to be Attached to Packaging Materials

The synthesis of PDA films will be done using a two-step procedure described by Silbert et al. [Silbert L, Shlush I B, Israel E, Porgador A, Kolusheva S, Jelinek R. 2006. Rapid Chromatic Detection of Bacteria by Use of a New Biomimetic Polymer Sensor. Applied and Environmental Microbiology. 72: 7339-7344]. The first-step comprises of creating vesicles using PDA monomers. These vesicles are then trapped to agar gels, before polymerizing the entire construct. More specifically, vesicles containing DMPC and 10,12-tricosadiynoic acid (2:3 molar ratio) will be prepared at a concentration of 1 mM. The lipids will then be dried together in vacuo. Following evaporation, distilled water will be added and the suspension will then be probe sonicated at 70° C. The resultant vesicle solution will be cooled at 4° C. overnight and then polymerized by irradiation at 254 nm for 0.5 minutes.

A chromatic lipid-PDA agar matrix is then prepared as follows. Unpolymerized PDA vesicles at a concentration of 5 mM will be added right after the sonication stage to hot LB agar. The mixture will then be cooled to room temperature. After solidification of the agar, the plate is kept at 4° C. for 2 days and polymerized by irradiation (254 nm, 40 s) in a UV cross-linker (UV-8000; Stratagene, Calif.).

Four different types of bacteria namely Serratia spp (gram −ve), Pseudomonas (gram −Ve), Staphylococcus aureus (gram +ve), and Staphylococcus pneumoniae (gram +ve) and fusarium (fungi) which are commonly associated to keratitis are used to challenge the PDA film sensors. Different concentrations of bacteria/fungi are spiked into the lens solutions to determine the detection limit and detection range. More specifically, bacterial samples are purchased from America Type Culture Collection (ATCC) and cultured as per provider specifications. A mounted digital camera is used to acquire images of PDA films in the presence of different concentrations of bacteria/fungi every 30 minutes for a period of 10 hours. Images are evaluated to calculate the sensor response time to bacteria/fungal contamination. The minimum detection capabilities of the film is also evaluated.

The PDA/vesicle films are further evaluated for stability in multipurpose contact lens solution at different temperature and pH. More specifically, PDA films are stored in the contact lens solution for a period 60 days. The films are also exposed to temperature and pH fluctuations. The PDA film storage lens solutions is then compared to normal lens solutions using mass spectroscopy to determine any constitutional changes which would indicate film leeching or degradation. In order to determine the stability of the PDA films mass spectroscopy is used to evaluate and obtain the chemical signatures of contact lens solutions. The chemical signatures of the bottled solution is compared with the signature obtained from the PVA film storage solution to detect PDA or agar leeching/degradation. The films are subjected to high temperature and pH fluctuations to evaluate their stability.

1. A packaging material for detection of a microbial product in a sample comprising a polydiacetylene (PDA) and a packaging polymer, wherein the microbial product contacts the PDA and a change of PDA color indicates detection.

2. The packaging material of claim 1, wherein a PDA solution is coated onto the packaging material.

3. The packaging material of claim 2, wherein the PDA solution is coated by dipping.

4. The packaging material of claim 2, wherein the PDA solution is coated by aerosolization.

5. The packaging material of claim 3, wherein the PDA solution comprises 10,12-tricosadiynoic acid, tetraethyl orthosilicate, nitric acid, tetrahydrofuran, and water, and wherein the mole ratios of the 10,12-tricosadiynoic acid, tetraethyl orthosilicate, tetrahydrofuran, nitric acid, and water are 1:9:312:0.13:0.05, respectively.

6. (canceled)

7. The packaging material of claim 1, wherein the PDA is incorporated into the packaging material, and wherein the composition is made by a process comprising mixing a PDA monomer and a packaging monomer prior to polymerization, curing and molding of the composition and wherein the material is formed by a process comprising the steps of: 1) dissolution of a PDA monomer in an aqueous solution to result in formation of a PDA vesicle (first solution), 2) dissolution of a packaging monomer in a mild organic solvent (second solution), 3) mixing the first and second solutions to form a third solution, and 4) polymerizing the third solution.

8. (canceled)

9. The packaging material of claim 1, further comprising a silica.

10. The packaging material of claim 9, wherein the material is prepared by a process comprising mixing a PDA monomer, a silica precursor, and a packaging monomer, wherein the material is prepared by a process comprising the steps of: 1) mixing a PDA monomer with a silica precursor (first solution), 2) mixing the first solution with a packaging monomer to form a second solution, and 3) polymerizing the second solution; or wherein the material is prepared by a process comprising the steps of: 1) mixing a PDA monomer, a silica precursor and a packaging monomer, and 2) polymerizing the mixture.

11. (canceled)

12. (canceled)

13. A fluid container comprising the packaging material, wherein the packaging material includes a polydiacetylene (PDA) and a packaging polymer, wherein the microbial product contacts the PDA and a change of PDA color indicates detection.

14. The fluid container of claim 13, wherein a material comprising the PDA is attached to an interior container wall surface.

15. The fluid container of claim 13, wherein an agarose gel comprising the PDA is placed on an interior container wall surface or an interior container cap surface.

16. A method for detecting one or more microbial products in a sample comprising, contacting the sample with a polydiacetylene (PDA), wherein a color change in the PDA indicates detection, wherein the sample is disposed within a container, wherein the container comprises a wall, an interior, and a cap, and wherein the container wall comprises the PDA.

17. The method of claim 16, wherein the PDA is incorporated into the container wall, wherein the container wall is formed by a process comprising mixing a PDA monomer with a packaging monomer and inducing polymerization of the monomers, and wherein the container wall is formed by a process comprising the steps of: 1) dissolution of a PDA monomer in an aqueous solution to result in formation of a PDA vesicle (first solution), 2) dissolution of a packaging monomer in a mild organic solvent (second solution), 3) mixing the first and second solutions to form a third solution, and 4) polymerizing the third solution.

18. (canceled)

19. (canceled)

20. The method of claim 16, wherein the container wall is formed by a process comprising mixing a PDA monomer, a silica precursor, and a packaging monomer, wherein the steps include: 1) mixing a PDA monomer with a silica precursor (first solution), 2) mixing the first solution with a packaging monomer to form a second solution, and 3) polymerizing the second solution; or wherein the steps include: 1) mixing a PDA monomer, a silica precursor and a packaging monomer, and 2) polymerizing the mixture.

21. (canceled)

22. (canceled)

23. The method of claim 16, wherein a PDA solution is coated onto an interior surface of the container wall, wherein the PDA is coated using a dipping process; or wherein a material comprising the PDA is attached to an interior container wall surface; or wherein an agarose gel comprising the PDA is placed on an interior container wall surface or an interior cap surface.

24. (canceled)

25. (canceled)

26. (canceled)
